(19) United States
(12) Patent Application Publication
(10) Pub. No.: US 2012/0207790 A1
(43) Pub. Date: Aug. 16, 2012

(54) IMMUNOSUPPRESSING AGENT COMPRISING MESENCHYMAL STEM CELL DERIVED FROM ADIPOSE TISSUE, AND USE THEREOF

(75) Inventors: Shoichi Maruyama, Nagoya-shi (JP); Takenori Ozaki, Nagoya-shi (JP); Yosuke Saka, Nagoya-shi (JP); Kazuhiro Furuhashi, Nagoya-shi (JP); Naotake Tsuboi, Nagoya-shi (JP)

(73) Assignee: National University Corporation Nagoya University, Nagoya-shi (JP)

(21) Appl. No.: 13/500,114

(22) PCT Filed: Aug. 30, 2010
(86) PCT No.: PCT/JP10/64682

§ 371 Date: Apr. 4, 2012

(30) Foreign Application Priority Data
Oct. 8, 2009 (JP) 2009-233991

(51) Int. Cl. A61K 35/12; A61P 17/00; A61P 37/06; A61P 29/00; C12N 5/077; C12N 5/071

(52) U.S. Cl. 424/278.1; 435/366; 435/351; 435/350; 435/325; 435/352; 435/363; 435/375

(57) ABSTRACT

To provide an immunosuppressing agent having high therapeutic effect and uses thereof. The immunosuppressing agent includes adipose-derived mesenchymal stem cells obtained by low-serum culture.

### TECHNICAL FIELD

The present invention relates to an immunosuppressing agent, and more specifically to an immunosuppressing agent including adipose-derived mesenchymal stem cells, and uses thereof. This application claims the benefit of priority from prior Japanese Patent Application No. 2009-233991, filed Oct. 8, 2009, the entire contents of which are incorporated herein by reference.

### BACKGROUND ART

The attempt to reconstruct damaged tissues using multipotent stem cells, which are capable of differentiation into various cells, has been made on a global scale. For example, mesenchymal stem cells (MSCs), which are one of multipotent stem cells, are capable of differentiation into various cells such as osteocytes, chondrocytes, and cardiac muscle cells, and their clinical applications are attracting attention. There are examples of clinical application of marrow-derived mesenchymal stem cells, and their effectiveness has been validated. Non-Patent Document 1 reports that marrow-derived mesenchymal stem cells have immunosuppressive activity, and are effective for the prevention of graft versus host disease (GVHD) after marrow transplantation.

In the prior patent application, the research group of the present inventors reported that adipose-derived mesenchymal stem cells (also referred to as adipose-derived stem cells: ASCs, adipose-derived regeneration cells: ADRCs, adipose-derived mesenchymal stem cells: AT-MSCs, or AD-MSCs) are applicable to the treatment of ischemic diseases, kidney dysfunction, or injuries as their novel uses, and that the cell population prepared by low-serum culture is particularly suitable for these uses (Patent Document 1).

### PRIOR ART DOCUMENT

### Patent Document

[Patent Document 1] WO 2008/018450

### Non-Patent Document

[Non-Patent Document 1]

Katarina Le Blanc, Ida Rasmusson, Berit Sundberg, Cecilia Gotherstrom, Moustapha Hassan, Mehmet Uzunel and Olle Ringden, Treatment of severe acute graft-versus-host disease with third party haploidentical mesenchymal stem cells, The Lancet, Volume 363, Issue 9419, Pages 1439-1441.

### SUMMARY OF THE INVENTION

### Problems to be Solved by the Invention

The present invention is intended to provide an immunosuppressing agent achieving high therapeutic effect, and uses thereof

### Means for Solving the Problem

Adipose tissues are regarded more useful as the source of mesenchymal stem cells than bone marrow, and are expected to find clinical applications, because they can be collected in a large amount by a simple operation, and impose only a light burden on patients during collection. Under such circumstances, the inventors focused on low-serum culture as the method for preparing adipose-derived mesenchymal stem cells. They further studied diligently to create novel uses of the cells obtained by the culture method. The result of experiments at the cell level and animal experiments using various disease models have proved that a cell population prepared by low-serum culture shows markedly stronger immunosuppressive activity than that prepared by conventional method (more specifically, culture under high-serum conditions). In other words, it was found that an immunosuppressing agent achieving high therapeutic effect is provided through the use of a cell population prepared by low-serum culture. In addition, it was suggested that the immunosuppressing agent is effective for the treatment of scleroderma, systemic lupus erythematosus, nephritis, and inflammatory lung disorders. The results of further study revealed that the adipose-derived mesenchymal stem cells prepared by low-serum culture (LASCs) show markedly higher therapeutic effect than the adipose-derived mesenchymal stem cells prepared from conventional high-serum culture (HASCs). Also obtained was an astonishing finding that LASCs transform M1 macrophages into M2 macrophages to increase the number of M2 macrophages, which results in the achievement of marked therapeutic effect. In addition, it was found that the main active site of the administered LASCs is not local, more specifically, LASCs exert systemic action, indicating that LASCs are useful as an active ingredient of an immunosuppressing agent. The present invention described hereinafter is mainly based on these findings.

[1] An immunosuppressing agent comprising:

(1) cells proliferated by culturing a sedimented cell population, which has been segmented by centrifuging a cell population separated from adipose tissues at 800 to 1500 rpm for 1 to 10 minutes, under low-serum conditions, or

(2) cells proliferated by culturing a cell population separated from adipose tissues, under low-serum conditions.

[2] The immunosuppressing agent of [1], wherein the low-serum conditions comprise a serum concentration of 5% (V/V) or less in the culture solution.

[3] The immunosuppressing agent of [1] or [2], which is capable of promoting an increase of M2 macrophages.

[4] The immunosuppressing agent of [3], wherein the increase of M2 macrophages is caused by the result of transformation of M1 macrophages into M2 macrophages.

[5] The immunosuppressing agent of any one of [1] to [4], wherein the adipose tissues are human adipose tissues.

[6] The immunosuppressing agent of any one of [1] to [5], which is used for treating scleroderma, nephritis, systemic lupus erythematosus, or inflammatory lung disorder.

[7] The immunosuppressing agent of any one of [1] to [6], which is used for systemic administration.

[8] A method for preparing cells showing immunosuppressive action, comprising:

(1) culturing a sedimented cell population, which has been sedimented by centrifuging a cell population separated from adipose tissues at 800 to 1500 rpm for 1 to 10 minutes, or a cell population separated from adipose tissues, under low-serum conditions; and

(2) culturing the proliferated cells in the presence of a substance enhancing immunosuppressive activity.

[9] The preparation method of [8], wherein the substance enhancing immunosuppressive activity is one or more substances selected from the group consisting of interferon γ (IFN-γ), IL-1α, IL-1β, IL-6, IL-12, IL-18, TNF-α, and LPS (lipo-polysaccharide).

[10] An immunosuppressing agent comprising the cells prepared by the preparation method of [8] or [9].

[11] A therapy for autoimmune diseases comprising administering the immunosuppressing agent of any one of [1] to [5] and [10] to an autoimmune patient.

[12] A therapy for autoimmune diseases comprising administering any of the cells described in the following (1) to (3) to an autoimmune patient in a therapeutically effective amount:

(1) cells proliferated by culturing a sedimented cell population, which has been sedimented by centrifuging a cell population separated from adipose tissues at 800 to 1500 rpm for 1 to 10 minutes, under low-serum conditions;

(2) cells proliferated by culturing a cell population separated from adipose tissues, under low-serum conditions; or

(3) cells prepared by culturing the cells of (1) or (2) in the presence of a substance enhancing immunosuppressive activity.

### BRIEF DESCRIPTION OF THE DRAWINGS

FIG. 1 shows a method of experimental for studying lymphocyte proliferation inhibiting activity. Human peripheral blood lymphocytes stimulated with phytohemagglutinin (PHA) and human adipose-derived mesenchymal stem cells (ASCs) were cocultured, and the A lymphocyte proliferation inhibitory effect was examined.

FIG. 2 shows the comparison of lymphocyte proliferation inhibiting activity of human adipose-derived mesenchymal stem cells (ASCs). The human ASCs prepared by low-serum culture (in a culture medium containing 2% FBS and 10 ng/ml bFGF) significantly inhibited the proliferation of lymphocytes. No PHA stimulation: lymphocytes were incubated without PHA stimulation; PHA stimulation only: lymphocytes stimulated with PHA were incubated; PHA stimulation+ASCs by high-serum culture: lymphocytes stimulated with PHA and human ASCs prepared by high-serum culture (in a culture medium containing 20% FBS and 10 ng/ml bFGF) were cocultured; PHA stimulation+ASCs by low-serum culture: lymphocytes stimulated with PHA and human ASCs prepared by low-serum culture (in a culture medium containing 2% FBS and 10 ng/ml bFGF) were cocultured. The graph ordinate shows the intake capacity for thymidine.

FIG. 3 shows the therapeutic effect of low-serum culture ASCs (LASCs) against scleroderma: (a) shows the comparison of the corium thickness; (b) shows the result of immunostaining; and (c) shows the result of quantitation of antinuclear antibodies, wherein the ordinate shows the antibody titer. In the scleroderma model, LASCs inhibited the autoantibody production, and improved the status of skin. As a result of the addition of IFN-γ induction (LASCs+IFN), the immunosuppressive activity of LASCs was enhanced. CONT represents the control group.

FIG. 4 shows the therapeutic effect of low-serum culture ASCs (LASCs) on systemic lupus erythematosus. The cumulative survival rates of the study groups and control group (CONT group) were compared. The abscissa shows the age of mice in week, and the ordinate shows the cumulative survival rate of mice.

FIG. 5 shows the therapeutic effect of low-serum culture ASCs (LASCs) against crescentic glomerulonephritis. The number of ED-1 positive cells (in 100 glomeruli) were compared. LASCs remitted glomerulitis in the nephritis model.

FIG. 6 shows the therapeutic effect of low-serum culture ASCs (LASCs) against crescentic glomerulonephritis. Comparison was made with the therapeutic effect of high-serum culture ASCs (HASCs). The blood urea nitrogen (BUN) (left), creatinine level (Cre) (center), and urinary protein (right) were compared between the control group (CONT), the group dosed with high-serum culture ASCs (HASCs), and the group dosed with low-serum culture ASCs (LASCs).

FIG. 7 shows the therapeutic effect of low-serum culture ASCs (LASCs) against crescentic glomerulonephritis. The kidney weight on day 7 was compared between the control group (CONT, n=10), the group dosed with high-serum culture ASCs (HASCs, n=7), and the group dosed with low-serum culture ASCs (LASCs, n=8). The left graph shows the comparison of kidney weight. The right photograph shows the comparison of the kidney size of the control group (CONT) and the group dosed with low-serum culture ASCs (LASCs).

FIG. 8 shows the therapeutic effect of low-serum culture ASCs (LASCs) against crescentic glomerulonephritis. The kidney tissues were stained with PAS on day 7, and the rate of glomerular crescents was compared between the control group (CONT), the group dosed with high-serum culture ASCs (HASCs), and the group dosed with low-serum culture ASCs (LASCs). The lower right graph shows the comparison of the rate of glomerular crescents.

FIG. 9 shows the therapeutic effect of low-serum culture ASCs (LASCs) against crescentic glomerulonephritis. The degree of deposition of anti-GBM IgG in the kidney tissues was compared between the control group (CONT), the group dosed with high-serum culture ASCs (HASCs), and the group dosed with low-serum culture ASCs (LASCs).

FIG. 10 shows the therapeutic effect of low-serum culture ASCs (LASCs) against crescentic glomerulonephritis. On day 7, the numbers of the ED-1 positive cells (M1 macrophages) and ED-2 positive cells (M2 macrophages) were individually counted, and compared between the control group (CONT), the group dosed with high-serum culture ASCs (HASCs), and the group dosed with low-serum culture ASCs (LASCs). The upper row shows immunostaining images, and the lower row shows the graphs of the quantitation results, wherein ns represents no significant difference.

FIG. 11 shows the therapeutic effect of low-serum culture ASCs (LASCs) against crescentic glomerulonephritis. The IL10 concentration in the renal cortex was compared between the control group (CONT), the group dosed with high-serum culture ASCs (HASCs), and the group dosed with low-serum culture ASCs (LASCs): the left graph shows the IL10 concentration, and the right graph represents the correlation between the IL10 concentration and the number of ED2-positive cells.

FIG. 12 shows the therapeutic effect of low-serum culture ASCs (LASCs) against inflammatory lung disorder. The body weight rate of change of the acute lung barrier models was compared between the control group (CONT), the group dosed with high-serum culture ASCs (HASCs), and the group dosed with low-serum culture ASCs (LASCs), wherein ns represents no significant difference.

FIG. 13 shows the therapeutic effect of low-serum culture ASCs (LASCs) against inflammatory lung disorder. The survival rate of the acute lung barrier models was compared between the control group (CONT) and the group dosed with low-serum culture ASCs (LASCs), and evaluated by the Kaplan-Meier method, wherein p<0.05 (by Cox-Mantel (Logrank) test)

### DESCRIPTION OF EMBODIMENTS

The present invention relates to an immunosuppressing agent and uses thereof. The immunosuppressing agent of the present invention contains adipose-derived mesenchymal stem cells prepared by low-serum culture (in the present description, “adipose-derived mesenchymal stem cells” and “adipose-derived mesenchymal stem cells obtained by low-serum culture” may be abbreviated as “ASCs” and “low-serum culture ASCs” or “LASCs”, respectively). In the present invention, “adipose-derived mesenchymal stem cells (ASCs)” means somatic stem cells contained in adipose tissues, and also includes the cells obtained by incubation of somatic stem cells (including subculture), as long as they have multipotency.

As shown by the below-described examples, the results of the study by the inventors indicate that LASCs have activity of promoting the increase of M2 macrophages (increasing the number of M2 macrophages). Therefore, the immunosuppressing agent of the present invention is characterized by this action. It was also found that LASCs cause transformation of M1 macrophages into M2 macrophages. On the basis of the fact, the immunosuppressing agent of the present invention is also characterized in that it “promotes the increases of M2 macrophages” and that “the increase of M2 macrophages is caused by the result of transformation of M1 macrophages into M2 macrophages.

In usual, low-serum culture ASCs are prepared from adipose tissues separated from a living body, and prepared into cells composing a cell population (including adipose-derived cells other than ASCs) in an “isolated state”. The “isolated state” means that the cells have been removed from its natural state (components of the living body), or the cells have been changed from their natural state by artificial operation. Adipose-derived mesenchymal stem cells may be also referred to as ADRC (adipose-derived regeneration cells), AT-MSC (adipose-derived mesenchymal stem cells), AD-MSC (adipose-derived mesenchymal stem cells), or the like. In the present description, the following terms, or adipose-derived mesenchymal stem cells, ASCs, ADRCs, AT-MSCs, and AD-MSCs may be substituted with each other.

### (Preparation of Low-Serum Culture ASCs)

The low-serum culture ASCs are obtained by culturing, under low-serum conditions, the stem cells separated and prepared from a fat matrix. The method for preparing ASCs to be cultured in a low-serum culture is not particularly limited. For example, ASCs may be prepared according to any known method (for example, Fraser J K et al. (2006) “Fat tissue: an underappreciated source of stem cells for biotechnology.” Trends in Biotechnology; April; 24(4): 150-4. Epub 2006 Feb 20. Review; Zuk P A et al. (2002) “Human adipose tissue is a source of multipotent stem cells.” Molecular Biology of the Cell; December; 13(12): 4279-95; Zuk P A et al. (2001) “Multilineage cells from human adipose tissues: implications for cell-based therapies.” And Tissue Engineering; April; 7(2): 211-28.). Apparatus for preparing ASCs from adipose tissues (for example, Celution (registered trademark) apparatus (Cytori Therapeutics Inc., San Diego, U.S.) are commercially available, and any of the apparatus may be used for the preparation of ASCs. The use of the apparatus allows the separation of the cell surface marker CD29 and CD44-positive cells from the adipose tissues. A specific example of the method for preparing low-serum culture ASCs is described below.

(1) Preparation of Population of Cells From Adipose Tissue

Adipose tissue can be obtained from an animal by means such as excision and suck. The term “animal” herein includes human and non-human mammalians (pet animals, domestic animal, and experimental animal. Specifically examples include mouse, rat, guinea pig, hamster, monkey, cow, pig, goat, sheep, dog, cat, and the like). In order to avoid the problem of immunological rejection, it is preferable that adipose tissue is collected from the same individuals as subjects (recipients) to which the immunosuppressing agent of the present invention is to be administered. However, adipose tissue of the same kinds of animals (other animals) or adipose tissue heterogeneous animals may be used.

An example of adipose tissue can include subcutaneous fat, offal fat, intramuscular fat, and inter-muscular fat. Among them, subcutaneous fat is a preferable cell source because it can be collected under local anesthesia in an extremely simple and easy manner and therefore the burden to a patient in collection is small. In general, one kind of adipose tissue is used, but two kinds or more of adipose tissues can be used. Furthermore, adipose tissues (which may not be the same kind of adipose tissue) collected in a plurality of times may be mixed and used in the later operation. The collection amount of adipose tissue can be determined by considering the kind of donors or kinds of tissue, or the necessary amount of ASCs. For example, the amount can be from 0.5 g-500 g. When a donor is human, it is preferable that the collection amount at one time is about 10-20 or less by considering a burden to the donor. The collected adipose tissue is subjected to removal of blood components attached thereto and stripping if necessary and thereafter, subjected to the following enzyme treatment. Note here that by washing adipose tissue with appropriate buffer solution or culture solution, blood components can be removed.

The enzyme treatment is carried out by digesting adipose tissue with protease such as collagenase, trypsin and Dispase. Such an enzyme treatment may be carried out by techniques and conditions that are known to a person skilled in the art (see, for example, R. I. Freshney, Culture of Animal Cells: A Manual of Basic Technique, 4th Edition, A John Wiley & Sones Inc., Publication). Preferably, enzyme treatment is carried out by the bellow-mentioned techniques and conditions.

A cell population obtained by the above-mentioned enzyme treatment includes multipotent stem cells, endothelial cells, interstitial cells, blood corpuscle cells, and/or precursor cells thereof. The kinds or ratios of the cells constituting the cell population depend upon the origin and kinds of adipose tissue to be used.

(2) Obtaining of Sedimented Cell Population (SVF Fraction: Stromal Vascular Fractions)

The cell population is then subjected to centrifugation. Sediments obtained by centrifugation are collected as sedimented cell population (also referred to as “SVF fraction” in this specification). The conditions of centrifugation are different depending upon the kinds or amount of cells. The centrifugation is carried out for example, at 800-1500 rpm for 1-10 minutes. Prior to the centrifugation, cell population after enzyme treatment can be subjected to filtration and tissue that has not been digested with enzyme contained therein can be removed.

The “SVF fraction” obtained herein includes ASCs. The kinds or ratio of cells constituting the SVF fraction depend upon the origin and kinds of adipose tissue to be used, conditions of the enzyme treatment, and the like. The SVF fraction is characterized by including CD34 positive and CD45 negative cell population, and that CD34 positive and CD45 negative cell population (International Publication WO2006/006692A1).

(3) Selective Culture of Adherent Cells (ASCs) and Cell Collection

The SVF fraction contains ASCs, and other cell components (endothelial cells, interstitial cells, erythroid cells, and precursor cells thereof). According to one aspect of the present invention, the following selective culture is carried out, and unnecessary cell components are removed from the SVF fraction. The cells thus obtained are used as ASCs and are subjected to a low-serum culture.

Firstly, the SVF fraction is suspended in an appropriate culture medium, seeded in a culture dish, and incubated overnight. Floating cells (non-adherent cells) are removed by exchanging the culture medium. Thereafter, culture is continued with the culture medium replaced as appropriate (for example, once every three days). Subculture is carried out as necessary. The passage number is not particularly limited. The culture medium may be selected from ordinary media used for culture of animal cells. Examples of the culture medium include Dulbecco's modified Eagle's Medium (DMEM) (NISSUI PHARMACEUTICAL, etc), α-MEM (Dainippon Seiyaku, etc), DMED: Ham's F12 mixed medium (1:1) (Dainippon Seiyaku, etc), Ham's F12 medium (Dainippon Seiyaku, etc.), and MCDB201 medium (Research Institute for the Functional Peptides). The culture medium may contain serum (for example, fetal bovine serum, human serum, or sheep serum) or serum replacement (for example, knockout serum replacement (KSR)). The amount of the serum or serum replacement may be adjusted in the range of, for example, 5% (v/v) to 30% (v/v).

The adherent cells are selectively survived and proliferated by the above-described operation. Next, the cells proliferated are collected. The cells may be collected by routine procedures and, for example, collected easily by enzyme treatment (treatment with trypsin or Dispase) and then cells are scraped out by using a cell scraper, a pipette, or the like. Furthermore, when sheet culture is carried out by using a commercially available temperature sensitive culture dish, cells may be collected in a sheet shape without carrying out enzyme treatment. The cells (ASCs) thus collected are used for the preparation of an immunosuppressing agent which contains effective cells at high purity.

(4) Low-serum culture (selective culture in a low-serum culture medium) and cell collection

Subsequently, low-serum culture is carried out. In the low-serum culture, the SVF fraction (when this step is carried out after (3), the cells collected in (3) are used) is incubated under low-serum conditions, and the desired multipotential stem cells (or ASCs) are selectively proliferated. Since the amount of serum to be used is small in the low-serum culture method, it is possible to use the serum of the subjects (recipients) themselves to which the immunosuppressing agent of the present invention is administered. That is to say, culture using autoserum can be carried. By using autoserum, it is possible to provide a immunosuppressing agent capable of excluding heterogeneous animal materials from manufacturing process and being expected to have high safety and high therapeutic effect. The “under low-serum conditions” herein denotes conditions in which a medium contains not more than 5% serum. Preferably, the sedimented cell population is cultured in a culture solution containing not more than 2% (V/V) serum. More preferably, the cells are cultured in a culture solution containing not more than 2% (V/V) serum and 1-100 ng/ml of fibroblast growth factor −2.

The serum is not limited to fetal bovine serum. Human serum, sheep serum, and the like, can be used. Preferably, the human serum, more preferably the serum of a subject to whom the immunosuppressing agent of the present invention is to be administered (that is to say, autoserum) is used.

As the medium, a medium for culturing animal cells can be used on condition that the amount of serum contained in the use is low. For example, Dulbecco's modified Eagle's Medium (DMEM) (NISSUI PHARMACEUTICAL, etc.), α-MEM (Dainippon Seiyaku, etc.), DMED: Ham's:F12 mixed medium (1:1) (Dainippon Seiyaku etc.), Ham's F12 medium (Dainippon Seiyaku, etc.), MCDB201 medium (Research Institute for the Functional Peptides), and the like, can be used.

By culturing by the above-mentioned method, ASCs can be selectively proliferated. Furthermore, since the ASCs proliferated in the above-mentioned culture conditions have a high proliferation activity, it is possible to easily prepare cells necessary in number for the immunosuppressing agent of the present invention by subculture. Note here that cells selectively proliferated by low-serum culture of SVF fraction is CD13, CD90 and CD105 positive and CD31, CD34, CD45, CD106 and CD117 negative (International Publication WO2006/006692A1).

Subsequently, the cells selectively proliferated by the above-described low-serum culture are collected. The collecting operation may be carried out in the same manner as in (3). The cells (ASCs) thus collected are used for the preparation of an immunosuppressing agent which contains effective cells at high purity.

The cell population (SVF fraction) obtained in the step (2) may be directly subjected to a low-serum culture. In this embodiment, the step (3) (selective culture) is omitted. Alternatively, the cells obtained by culture under low-serum conditions after several rounds of subculture of the SVF fraction by a conventional method (under high-serum under conditions) are also useful as low-serum culture ASCs.

In order to enhance immunosuppressive activity, as an additional culture step, it is preferred that the cells proliferated in a low-serum culture be incubated in the presence of a substance which enhances immunosuppressive activity, such as interferon γ (IFN-γ), IL-1α, IL-1β, IL-6, IL-12, IL-18, TNF-α, or LPS (lipo-polysaccharide). More specifically, in a preferred embodiment, the cells proliferated in a low-serum culture are stimulated thereby enhancing the immunosuppressive activity, and used to prepare the immunosuppressing agent. The amount of the substance enhancing immunosuppressive activity (the amount added to the culture medium) may be established as appropriate. For example, the amount of IFN-γ is preferably from 100 to 1000 IU/mL. The period of culture (incubation time) is not particularly limited. For example, the culture is incubated for 1 to 24 hours. Other culture conditions may follow the low-serum culture conditions. The culture step is herein referred to as immunosuppressive activity enhancing step.

(5) Formulation

The cells obtained by the low-serum culture, or the cells obtained after the immunosuppressive activity enhancing step are suspended in a saline solution or an appropriate buffer solution (for example, phosphate buffer solution), thereby preparing an immunosuppressing agent. In order to administer therapeutically effective amount of cells, each dose preferably contains, for example, 1×10⁶ to 1×10¹⁰ cells. The cell content may be adjusted as appropriate in consideration of the intended use; disease to be treated; sex, age, and body weight of the subject (recipient); condition of the affected skin; and condition of the cells.

The immunosuppressing agent of the present invention may include, for example, dimethylsulfoxide (DMSO), serum albumin, and the like, for protecting the cells; antibiotic and the like for inhibiting contamination of bacteria; various components(vitamins, cytokines, growth factors steroids and the like) for activation, proliferation or differentiation of cells. Furthermore, the immunosuppressing agent of the present invention may contain pharmaceutically acceptable other components (for example, carrier, excipient, disintegrating agents, buffer, emulsifier, suspension, soothing agent, stabilizer, preservatives, antiseptic, physiologic saline, etc.).

In the above-mentioned method, the immunosuppressing agent is formed by using cells proliferated by low-serum culture of SVF fraction. However, the immunosuppressing agent may be directly formed by the low-serum culture of cell population obtained from adipose tissue (without carrying out centrifugation for obtaining SVF fraction). That is to say, in one embodiment of the present invention, cells proliferated by the low-serum culture of cell population obtained from adipose tissue are used as low-serum culture ASCs. In the present embodiment, it is preferred that the above-described immunosuppressive activity enhancing step be carried out after the low-serum culture, thereby enhancing the immunosuppressive activity of the ASCs.

### (Applicable Diseases)

The immunosuppressing agent of the present invention is useful for various purposes wherein immunosuppression brings about preventive or therapeutic effect. Typically, the immunosuppressing agent of the present invention is useful for the purpose of treatment of autoimmune diseases. Examples of the autoimmune disease to be treated include nephritis (for example, ANCA-related nephritis, antiglomerular basement-membrane antibody nephritis, rapidly progressive glomerulonephritis, IgA nephritis, purpura nephritis, membranous glomerulonephritis, membranoproliferative glomerulonephritis, focal glomerulosclerosis, thrombogenic glomerulus nephritis, minute change nephrotic syndrome, and other nephrotic syndromes), hepatitis (for example, autoimmune hepatitis), collagen diseases (for example, scleroderma, systemic lupus erythematosus, articular rheumatism, skin plot flame, Sjögren syndrome, mixed collagen disease), inflammatory lung disorders (for example, chronic obstructive pulmonary diseases, interstitial pneumonia, and acute lung disorders), vasculitis (Takayasu disease and periarteritis nodosa), Behcet's disease, and sarcoidosis. In the same manner as conventional immunosuppressing agents, the immunosuppressing agent of the present invention may be used for the purpose of prevention of graft versus host disease (GVHD), which is a complication of transplantation. Alternatively, the immunosuppressing agent of the present invention may be used for the purpose of experiment or investigation for confirming or verifying the effect of the agent.

### (Application Objects)

The typical target of the immunosuppressing agent of the present invention is human. However, the immunosuppressing agent may be formulated for mammals other than human (including pet animals, livestock, experimental animals, such as mice, rats, guinea pigs, hamsters, monkeys, bovines, pigs, goats, sheep, dogs, and cats).

### (Administration Method)

The administration route of the immunosuppressing agent of the present invention is not particularly limited. The immunosuppressing agent of the present invention is administered by, for example, intravenous injection, intraarterial injection, intraportal injection, intradermal injection, subcutaneous injection, intramuscular injection, or intraperitoneal injection. The agent may be administered systemically or locally. Examples of local administration include direct injection into the target tissue, organ, or apparatus. The administration schedule may be made in consideration of the sex, age, body weight, and clinical condition of the subject (patient). The agent may be administered as single dose, or multiple doses continuously or periodically. The interval of multiple-dose administration is not particularly limited and may be, for example, one day to three months. The number of administration is also not particularly limited. The number of administration is for example, two to ten.

As shown by the below-described examples, the results of the study by the inventors indicate that the cells composing the immunosuppressing agent of the present invention (LASCs) acts systemically rather than locally. More specifically, it has been found that the cells exhibit preferred behavior as an active ingredient of the immunosuppressing agent. The fact also means that LASCs are particularly suitable for systemic administration (for example, intravenous injection).

Any of the following cells of (1) to (3) may be directly administered to autoimmune patients in a therapeutically effective amount.

(1) cells proliferated by culturing a sedimented cell population, which has been sedimented by centrifuging a cell population separated from adipose tissues at 800 to 1500 rpm for 1 to 10 minutes, under low-serum conditions;

(2) cells proliferated by culturing a cell population separated from adipose tissues under low-serum conditions; or

(3) cells prepared by culturing the cells of (1) or (2) in the presence of a substance enhancing immunosuppressive activity.

### EXAMPLES

Lymphocyte Proliferation Inhibiting Activity of Low-Serum Culture ASCs

1-1. Experimental Method

(1) Preparation of Human Low-Serum Culture ASCs

The SVF fraction was prepared from human adipose tissues by the following procedure. Firstly, subcutaneous adipose tissues were collected, during laparotomy, from a patient (50 years of age, female) from whom consent had been obtained prior to surgery. The adipose tissues were washed three times with 30 ml of DMEM/F12 medium (a mixture of equal amounts of Dulbecco's Modified Eagle's Medium and F12 culture medium, Sigma-Aldrich Co. LCC.), thereby removing adhered blood. Secondly, the adipose tissues were fragmented with a surgical knife in a sterilized culture dish. The adipose tissues were placed in a 50-ml centrifuge tube (Falcon), and weighed (about 1 g). 2 ml of 1 mg/ml collagenase type 1 (Worthington) solution was added in the above-described centrifuge tube, and then shaken for 1 hour at 37° C. and 120 rpm. Subsequently, 10 ml of DMEM/F12 medium was added in a centrifuge tube, and pipetted. The cell suspension after pipetting was filtered through a filter having a pore size of 100 μm (Falcon). The filtrate thus obtained was centrifuged at room temperature, 1200 rpm, for 5 minutes. The sediment was collected, and used as the SVF fraction.

The SVF fraction was subjected to a low-serum culture by the following procedure. 10⁵ nucleated cells in the SVF fraction were suspended in 5 ml of low-serum culture solution, and seeded in fibronectin-coated 25 cm² flask (Falcon). The low-serum culture solution was prepared as described below (a to e).

(a) 5.7 g of DMEM (NISSUI PHARMACEUTICAL), 7 g of MCDB201 (Sigma-Aldrich Co. LCC.), 0.35 g of L-glutamine (Sigma-Aldrich Co. LCC.), 1.2 g of NaHCO₃ (Sigma-Aldrich Co. LCC.), 1 ml of 0.1 mM ascorbic acid (Wako Pure Chemical Industries, Ltd.), and 0.5 ml of antibiotic (100,000 units/ml penicillin and 100 mg/ml streptomycin) were dissolved in 980 ml of distilled water.

(b) The pH was adjusted to 7.2 with 10N NaOH.

(c) The solution was filtered and sterilized.

(d) 10 ml of linoleic acid-albumin (Sigma-Aldrich Co. LCC.) and 10 ml of 100×ITS (10 mg of insulin, 5.5 mg of transferrin, and 5 μg of sodium selenite, Sigma-Aldrich Co. LCC.) were added.

(e) Fetal bovine serum FBS (Gibco) and bFGF (PeproTech) were added (final concentration of FBS: 2%, final concentration of bFGF: 10 ng/ml).

The whole medium was replaced every two days. After the cells became confluent, they were washed with PBS containing 1 mM EDTA, and treated with 0.05 to 0.25% trypsin solution thereby removing and collecting the cells, and the collected cells were seeded on a fibronectin-coated plate (made using human fibronectin of Sigma-Aldrich Co. LCC.) at a density of 4000 cells/cm² in the same manner as described above. The subculture was repeated as necessary (cells of the fourth to fifth passage were used in the present experiment).

For comparison, the cells prepared from a bFGF-free high-serum culture (incubated in a medium containing 20% FBS and no bAFGF) and the cells prepared by high-serum culture (in a medium containing 20% FBS and 10 ng/ml bFGF) were provided.

(2) Study of Lymphocytes Proliferation Inhibiting Activity

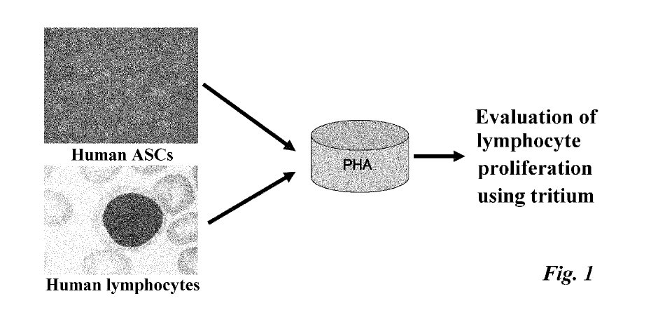

Phytohemagglutinin (PHA) (20 μg/mL) was added to the lymphocytes collected and separated from human peripheral blood, thereby stimulating the lymphocytes and promoting the proliferation. In order to study the lymphocytes proliferation inhibitory effect, the human ASCs obtained by the above-described three culture methods were coincubated with the lymphocytes in the proportion of 1/15. After the culture, the number of lymphocytes was counted, using the uptake of thymidine labeled with tritium as the index. (FIG. 1).

1-2. Result

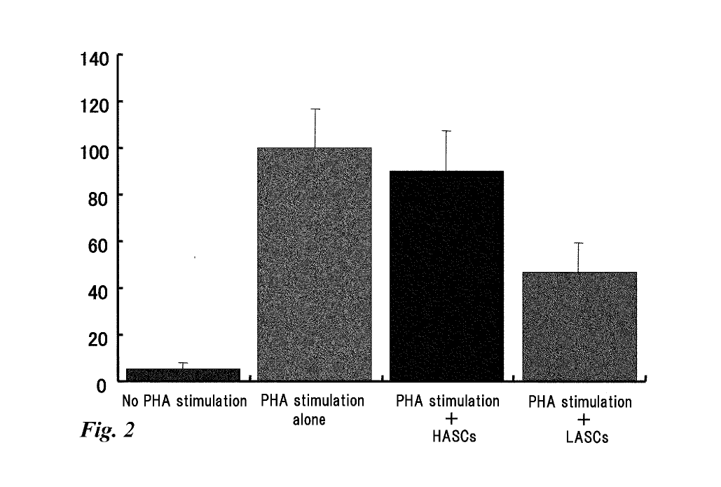

The ASCs prepared by low-serum culture (low-serum culture ASCs) showed markedly higher lymphocyte proliferation inhibitory effect than the ASCs prepared by bFGF-free high-serum culture or high-serum culture (FIG. 2).

2. Therapeutic Effect of Low-Serum Culture ASCs Against Scleroderma

2-1. Experimental Method

(1) Modeling of Scleroderma

Balb/c mice (6 weeks old, female, CLEA Japan Inc.) were used in the experiment. Bleomycin was administered to the mice by subcutaneous injection five days a week for three weeks, thereby inducing scleroderma.

(2) Cell Preparation

The subcutaneous fat was collected from the Balb/c mice (6 weeks old, female) and prepared into SVF, and incubated in a low-serum culture (2% FBS). Cells of the fourth to fifth passage (low-serum culture ASCs; LASCs) were used in the experiment. The preparation of the SVF and low-serum culture were carried out in the same manner as in 1-1.

(3) Cell Administration

In the group dosed with LASCs, LASCs (0.3×10⁶ cells were diluted in 200 μl of PBS) were administered from the tail vein on day 6 and 13 after initiation of bleomycin administration. In order to prevent pulmonary infarction, LASCs was administered together with 5 μl of heparin. In the group dosed with LASCs+IFN, LASCs were stimulated with IFN-λ (added to the culture medium at a concentration of 500 units/ml) from 6 hours before the administration, the IFN-λ was completely removed by washing, and LASCs were administered in the same manner as in the group dosed with LASCs. After three weeks, these groups were euthanized, and the skin thickness and autoantibody were quantitatively evaluated.

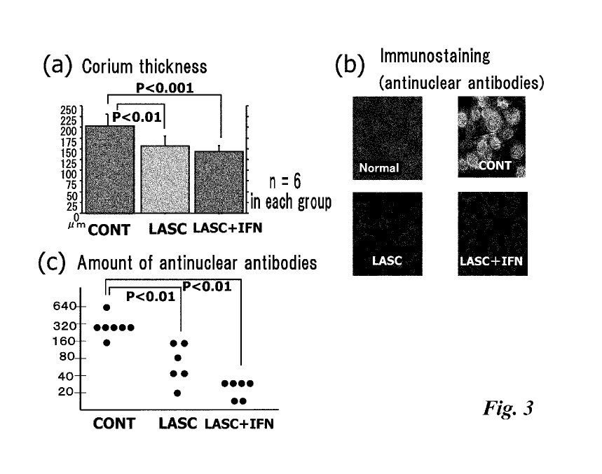

2-2. Result (FIG. 3)

Skin hardening was significantly ameliorated in both the group dosed with LASCs and the group dosed with LASCs+INF. The autoantibody production was inhibited in the LASC group, and more markedly inhibited in the LSC+INF group.

3. Therapeutic Effect of Low-Serum Culture ASCs Against Systemic Lupus Erythematosus

3-1. Experimental Method

(1) Cell Preparation

SVF was prepared from the subcutaneous fat collected from male NZB/W·F1 mice (systemic lupus erythematosus model mice; CLEA Japan Inc.), and a portion of the SVF was subjected to a low-serum culture (2% FBS). The SVF preparation and low-serum culture were carried out in the same manner as in 1-1. Another portion of the SVF was subjected to a high-serum culture (incubated in a culture medium containing 20% FBS and 10 ng/ml bFGF). The cells obtained by low-serum culture (cells of the fourth to fifth passage) and the cells obtained by high-serum culture (cells of the fourth to fifth passage) were used in the experiment.

(2) Study of Effect of Improving the Survival Rate of Systemic Lupus Erythematosus Model Mice

24 female NZB/W·F1 mice (CLEA Japan, Inc.) were divided into two groups each containing 12 animals, and the following two types of ASCs were administered from the tail vein after the mice were 14 weeks old, once a week, eight times in total.

In the group dosed with LASCs, LASCs were stimulated with IFN-γ (added to the culture medium at a concentration of 500 units/ml) from 6 hours before the administration, and the IFN-γ was completely removed by washing. Every time 1.0×10⁶ cells were diluted in 300 μl of PBS, and the dilution was administered. In order to prevent pulmonary infarction, LASCs was administered together with 5 μl of heparin.

In the group dosed with high-serum culture ASCs, the cell preparation and administration were carried out in the same manner as in the group dosed with LASCs, except that the cells obtained by high-serum culture were used.

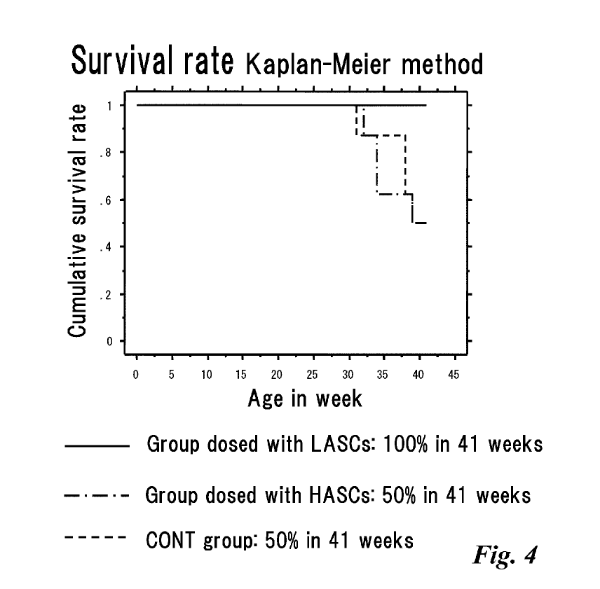

3-2. Result (FIG. 4)

When no cell was administered, 50% of the NZB/W·F1 mice died by the 41st week (CONT group). No therapeutic effect was found in the group dosed with high-serum culture ASCs. On the other hand, all the animals of the group dosed with LASCs were alive after a lapse of 41 weeks.

4. Therapeutic Effect of Low-Serum Culture ASCs Against Glomerulus Nephritis 1

4-1. Experimental Method

(1) Modeling of Crescentic Glomerulonephritis

WKY rats (8 weeks old, female, Charlesriver Laboratories Japan Inc.) were used in the experiment. TF78 (anti-GBM mouse IgG, Shigei Medical Research Institute) was administered intraperitoneally to the rats, thereby inducing crescentic glomerulonephritis.

(2) Cell Preparation

SVF was prepared from the subcutaneous fat collected from the WKY rats, and subjected to a low-serum culture (2% FBS). The cells of the fourth to fifth passage (low-serum culture ASCs; LASC) were used in the experiment. The SVF preparation and low-serum culture were carried out in the same manner as in 1-1.

(3) Study of Therapeutic Effect in Crescentic Glomerulonephritis Model

The 12 animals of the crescentic glomerulonephritis models were divided into three groups each containing four animals, and treated by the following procedure.

Group dosed with LASCs: On day 2 and 5 after administration of TF78, LASCs (2×10⁶ cells were diluted in 2 ml of PBS) were administered from the tail vein.

Group dosed with LASCs+IFN: LASCs were stimulated with IFN-γ (added to the culture medium at a concentration of 500 units/ml) from 6 hours before the administration, the IFN-γ was completely removed by washing, and LASCs were administered in the same manner as in the group dosed with LASCs.

Control group: On day 2 and 5 after administration of TF78, 2 ml of PBS was administered from the tail vein.

4-2. Result

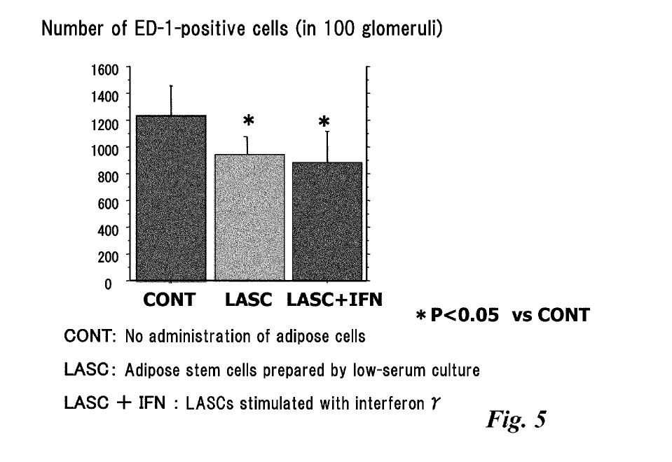

On day 7 after administration of TF78, the number of ED-1-positive cells (macrophages) was counted, thereby measuring the degree of inflammation in the glomerulus. The glomerulitis was significantly ameliorated in both the group dosed with LASCs and the group dosed with LASCs+INF (FIG. 5).

5. Therapeutic Effect of Low-Serum Culture ASCs Against Glomerulus Nephritis 2

5-1. Experimental Method

(1) Modeling of Crescentic Glomerulonephritis

WKY rats (8 weeks old, female, Charlesriver Laboratories Japan Inc.) were used in the experiment. TF78 (anti-GBM mouse IgG, Shigei Medical Research Institute) was administered intraperitoneally to the rats, thereby inducing crescentic glomerulonephritis.

(2) Cell Preparation

SVF was prepared from the subcutaneous fat collected from the WKY rats, and incubated in a low-serum culture (2% FBS). The cells of the fourth to fifth passage (low-serum culture ASCs; LASCs) were used in the experiment. As a comparative subject, the cells prepared by high-serum culture (20%) (high-serum culture ASCs; HASCs) were provided (prepared in the same manner as low-serum culture ASCs, except that high-serum culture was carried out in place of low-serum culture).

(3) Study of therapeutic effect in crescentic glomerulonephritis model

22 mice of the crescentic glomerulonephritis models were divided into three groups (8 mice of the group dosed with LASCs, 7 mice of the group dosed with HASCs, and 7 mice of the control group), and treated by the following procedure.

Group dosed with LASCs: on day 0, 1, 3, 4, and 5 after administration of TF78, LASCs (1×10⁶ cells were diluted in 2 ml of PBS) were intraperitoneally administered.

Group dosed with HASCs: on day 0, 1, 3, 4, and 5 after administration of TF78, HASCs (1×10⁶ cells were diluted in 2 ml of PBS) were intraperitoneally administered.

Control group: on day 0, 1, 3, 4, and 5, and on day 2 and 5 after administration of TF78, 2 ml of PBS was intraperitoneally administered.

5-2. Result

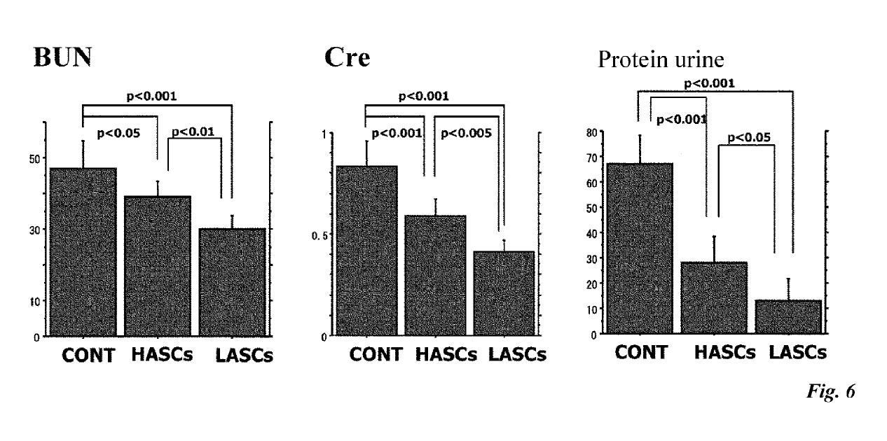

Kidney function (BUN and Cre) and protein urine were evaluated on day 7 and 5, respectively. The kidney function was not worse in the groups dosed with ASCs than in the control group, and the kidney protective effect was significantly strong in the group dosed with LASCs than in the group dosed with HASCs (FIG. 6). The protein urine was lower in the groups dosed with ASCs than in the control group, and the protein urine in the group dosed with LASCs was significantly lower than in the group dosed with HASCs (FIG. 6). It is worthy of note that the protein urine in the group dosed with LASCs was mostly within the normal range.

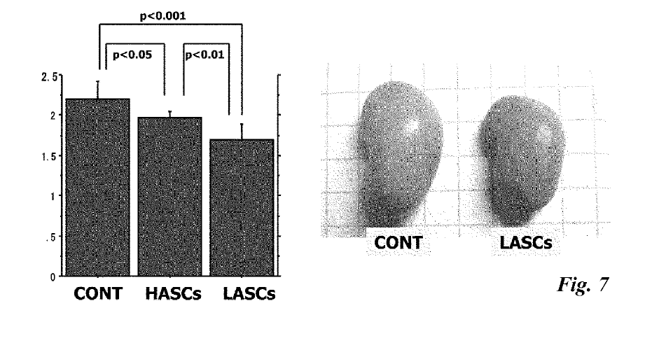

The both kidneys were weighed on day 7. The kidney weight was lighter in the groups administered with ASCs than in the control group, and was significantly lighter in the LASC group than in the HASC group (FIG. 7). These results indicate that the ASCs ameliorated the swelling caused by nephritis.

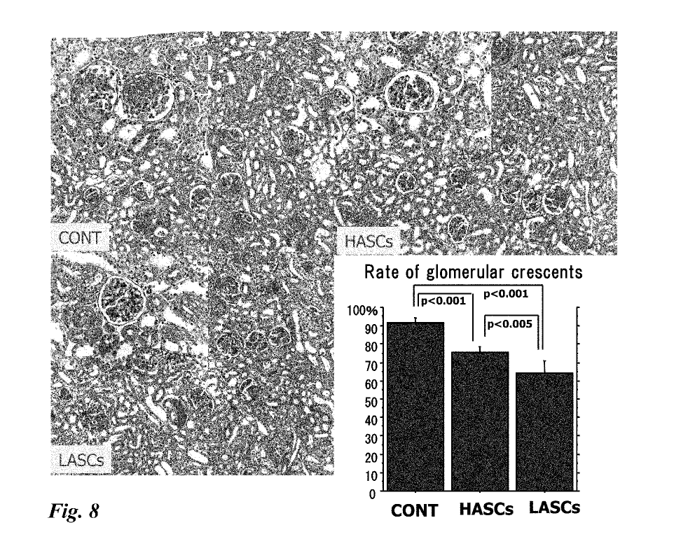

As a result of PAS staining of kidney tissues, the rate of glomerular crescents was lower in the groups dosed with ASCs than in the control group (FIG. 8). In addition, the rate of glomerular crescents was significantly lower in the LASC group than in the HASC group.

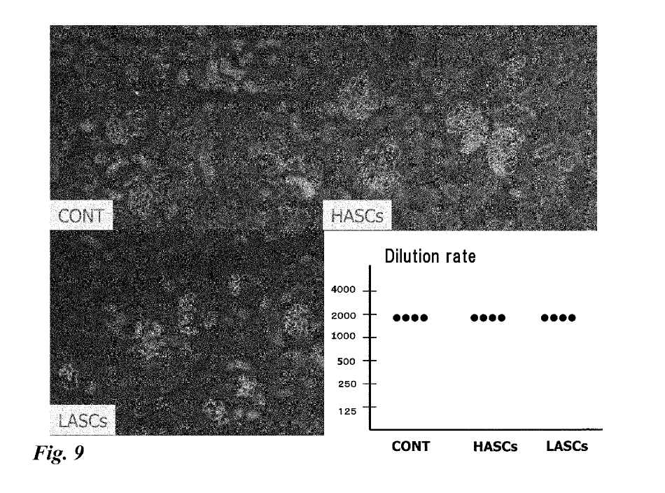

Deposition of anti-GBM IgG in the kidney tissues was compared, and no difference was found between the groups dosed with LASCs, HASCs, and control group (FIG. 9). More specifically, it was confirmed in vivo that the administered ASCs do not adsorb the antibody. The fact suggests that ASCs ameliorated inflammation reaction after deposition of the antibody.

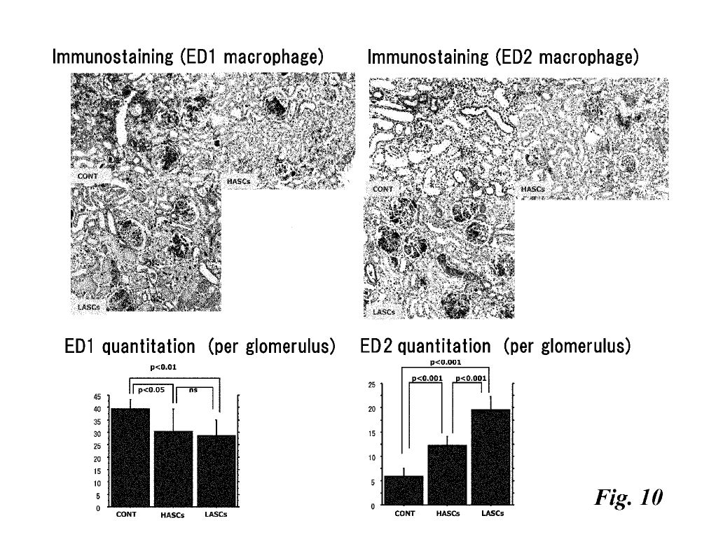

On day 7, the numbers of the ED-1 positive cells (M1 macrophages) and ED-2 positive cells (M2 macrophages) were individually counted. Interestingly, the number of the ED-2 positive cells, which are immuneregulatory macrophages (M2 macrophages), per glomerulus was higher in the groups dosed with ASCs than in the control group (FIG. 10). In addition, the number was highest in the group dosed with LASCs. The number of the ED-1 positive cells, which are inflammatory macrophages (M1 macrophages), was smaller in the groups dosed with ASCs than in the control group, and no difference was found between the group dosed with LASCs and the group dosed with HASCs (FIG. 10). These results suggest that LASCs strongly increase M2 macrophages. In consideration of these results, the action of LASCs was further studied. Specifically, intraperitoneal macrophages were collected from the WKY rats of the group dosed with LASCs, and coincubated together with GFP-positive LASCs for 48 hours in the presence of the antibody against M2 macrophages (the color of the cells transformed into M2 macrophages changes into red). As a result of observation, the red signal became stronger with time (the result is not shown), indicating that M1 macrophages are transformed into M2 macrophages. More specifically, it was confirmed that LASC transforms M1 macrophages into M2 macrophages.

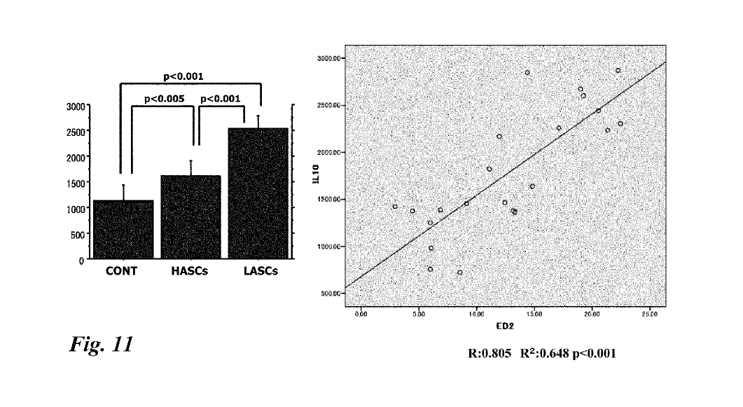

The IL10 concentration of the renal cortex was higher in the groups dosed with ASCs than in the control group, and highest in the group dosed with LASCs (FIG. 11, left). More specifically, a correlation was found between the IL10 concentration and the number of the ED2-positive cells (FIG. 11, right), and the IL10-producing cells are likely M2 macrophages.

6. Therapeutic Effect of Low-Serum Culture ASCs Against Inflammatory Lung Disorder

6-1. Experimental Method

Balb/c mice (6 weeks old, female, CLEA Japan Inc.) were used in the experiment. Bleomycin was endotracheally administered to the mice (acute lung disorder model). On day 0 after the treatment, low-serum culture ASCs (LASCs) prepared from the subcutaneous fat of the mice were administered (the group dosed with LASCs). For comparison, a group dosed with high-serum culture ASCs (HASCs) (group dosed with HASCs) and a group dosed with normal saline solution (control group) were provided. The body weight change and survival rate of these groups were recorded.

6-2. Result

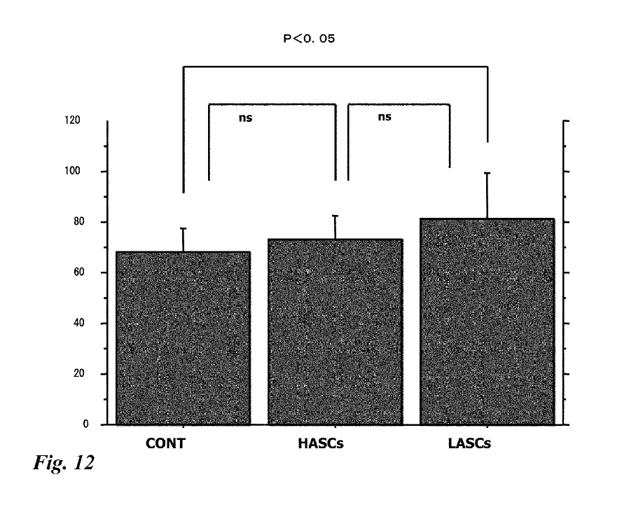

The body weight loss on day 9 as compared to on day 0 was compared. In comparison with the control group, the body weight loss was significantly inhibited in the group dosed with LASCs (FIG. 12). No significant difference was found between the group dosed with HASCs and the control group. It is known that the body weight change reflects the degree of lung disorder in the present model.

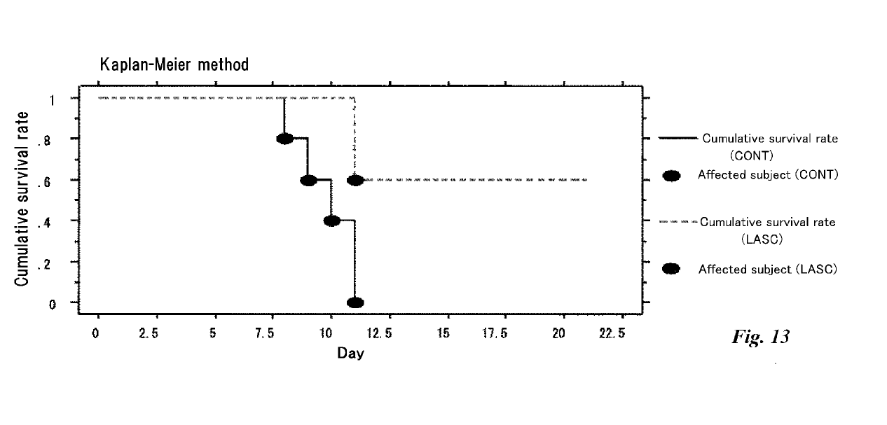

The comparison of the survival rate indicate that the survival rate is significantly improved in the group dosed with LASCs (FIG. 13).

7. Localization of Administered ASCs

The behavior of the intravenously administered ASCs was studied. The LASCs stained with CSFE were intravenously administered (once a day, four consecutive days), the tissues (kidney, lung, spleen, and lymph node) were collected on day 5, and ASCs were detected. As a result of this, only a slight number of ASCs was found in the kidney and lymph node (the result is not shown). On the other hand, the presence of many ASCs was found in the lung and spleen (the result is not shown). These results suggest that the administered ASCs do not directly act in the kidney, but act in the lung and spleen, and exert their effects (the increase of M2 macrophages and transformation of Ml macrophages into M2 macrophages).

8. Summary

These results suggest that low-serum culture ASCs (LASCs) inhibit lymphocyte proliferation, and are effective in the treatment of autoimmune diseases such as scleroderma, systemic lupus erythematosus, and nephritis. It was also revealed that LASCs show markedly higher therapeutic effect than high-serum culture ASCs (HASCs). Furthermore, it was found that LASCs transform Ml macrophages into M2 macrophages to increase the number of M2 macrophages, which results in the achievement of marked therapeutic effect. In addition, it was suggested that the main active site of the administered LASCs is not local, more specifically, LASCs exert systemic action.

### INDUSTRIAL APPLICABILITY

The immunosuppressing agent of the present invention is used for, for example, treatment of autoimmune diseases. Examples of the autoimmune disease to be treated include nephritis (ANCA-related nephritis, antiglomerular basement-membrane antibody nephritis, rapidly progressive glomerulonephritis, IgA nephritis, purpura nephritis, membranous glomerulonephritis, membranoproliferative 2 5 glomerulonephritis, focal glomerulosclerosis, thrombogenic glomerulus nephritis, minute change nephrotic syndrome, and other nephrotic syndromes), hepatitis (for example, autoimmune hepatitis), collagen diseases (for example, scleroderma, systemic lupus erythematosus, articular rheumatism, skin plot flame, Sjögren syndrome, mixed collagen disease), vasculitis (Takayasu disease and periarteritis nodosa), Behcet's disease, and sarcoidosis. The immunosuppressing agent of the present invention may be used for the purpose of prevention of graft versus host disease (GVHD), which is a complication of transplantation.

The present invention will not be limited to the above-described explanations of the embodiments and examples. Various modifications readily appreciated by those skilled in the art are also included in the present invention, without departing from the scope of the description of claims.

The articles, unexamined patent publications, and published unexamined patent applications cited in the present description are hereby incorporated by reference herein in their entirety.

1. An immunosuppressing agent comprising:
(1) cells proliferated by culturing a sedimented cell population, which has been sedimented by centrifuging a cell population separated from adipose tissues at 800 to 1500 rpm for 1 to 10 minutes, under low-serum conditions, or
(2) cells proliferated by culturing a cell population separated from adipose tissues, under low-serum conditions.

2. The immunosuppressing agent of claim 1, wherein the low-serum conditions comprises a serum concentration of 5% (V/V) or less in the culture solution.

3. The immunosuppressing agent of claim 1, which is capable of promoting an increase of M2 macrophages.

4. The immunosuppressing agent of claim 3, wherein the increase of M2 macrophages is caused by the result of transformation of M1 macrophages into M2 macrophages.

5. The immunosuppressing agent of any one of claim 1, wherein the adipose tissues are human adipose tissues.

6. The immunosuppressing agent of claim 1, which is used for treating scleroderma, nephritis, systemic lupus erythematosus, or inflammatory lung disorder.

7. The immunosuppressing agent of claim 1, which is used for systemic administration.

8. A method for preparing cells showing immunosuppressive action, comprising:
(1) culturing a sedimented cell population, which has been sedimented by centrifuging a cell population separated from adipose tissues at 800 to 1500 rpm for 1 to 10 minutes, or a cell population separated from adipose tissues, under low-serum conditions; and
(2) culturing the proliferated cells in the presence of a substance enhancing immunosuppressive activity.

9. The preparation method of claim 8, wherein the substance enhancing immunosuppressive activity is one or more substances selected from the group consisting of interferon γ (IFN-γ), IL-1αa, IL-β, IL-6, IL-12, IL-18, TNF-α, and LPS (lipo-polysaccharide).

10. An immunosuppressing agent comprising the cells prepared by the preparation method of claim 8.

11. A therapy for autoimmune diseases comprising administering the immunosuppressing agent of claim 1 to an autoimmune patient.

12. A therapy for autoimmune diseases comprising administering any of the cells described in the following (1) to (3) to an autoimmune patient in a therapeutically effective amount:
(1) cells proliferated by culturing a sedimented cell population, which has been sedimented by centrifuging a cell population separated from adipose tissues at 800 to 1500 rpm for 1 to 10 minutes, under low-serum conditions;
(2) cells proliferated by culturing a cell population separated from adipose tissues, under low-serum conditions; or
(3) cells prepared by culturing the cells of (1) or (2) in the presence of a substance enhancing immunosuppressive activity.
